(19) United States
(12) Patent Application Publication
(10) Pub. No.: US 2026/0083957 A1
(43) Pub. Date: Mar. 26, 2026

(54) CIRCULATORY SUPPORT SYSTEM WITH DIAPHRAGM PUMP

(71) Applicant: BOSTON SCIENTIFIC SCIMED, INC., Maple Grove, MN (US)

(72) Inventors: Tim O'Connor, Galway (IE); David Ryan, Mayo (IE); Gavin O'Brien, Dublin (IE); Chris Cullen, Galway (IE); Richard O'Sullivan, Turloughmore (IE); Benjamin Breidall, Eden Prairie, MN (US)

(73) Assignee: BOSTON SCIENTIFIC SCIMED, INC., Maple Grove, MN (US)

(21) Appl. No.: 19/333,640

(22) Filed: Sep. 19, 2025

Related U.S. Application Data
(60) Provisional application No. 63/697,204, filed on Sep. 20, 2024.

(51) Int. Cl. A61M 60/178; A61M 60/258; A61M 60/268; A61M 60/462; A61M 60/89

(52) U.S. Cl. CPC A61M 60/178; A61M 60/258; A61M 60/268; A61M 60/462; A61M 60/89

(57) ABSTRACT

Example medical devices including example cardiac pumps are disclosed. An example percutaneous circulatory support device system includes a blood pump including a pump housing having a distal end region and a proximal end region, a blood inlet positioned along the distal end region, a blood outlet positioned along the proximal end region and a first diaphragm pump positioned within the pump housing. Further, the first diaphragm pump is configured to draw blood into the pump inlet and pump blood out of the blood outlet.

### CROSS REFERENCE TO RELATED APPLICATIONS

The present application claims the benefit of U.S. Provisional Patent Application Ser. No. 63/697,204, filed Sep. 20, 2024, the disclosure of which is incorporated herein by reference.

### TECHNICAL FIELD

The present disclosure relates to percutaneous circulatory support device systems. More specifically, the disclosure relates to percutaneous circulatory support devices that include a diaphragm pump or piston pump.

### BACKGROUND

Percutaneous circulatory support devices such as blood pumps can provide transient cardiac support in patients whose heart function or cardiac output is compromised. Such devices may be delivered percutaneously from the femoral artery, retrograde through the descending aorta, over the aortic arch, through the ascending aorta across the aortic valve, and into the left ventricle. Some percutaneous circulatory support devices may include a blood pumping system configured to move blood from lower chambers of the heart to the rest of the body. An optimal blood pumping system should have a low profile and be flexible enough to facilitate percutaneous access and tracking through tortuous anatomy. Further, the blood pumping system should be configured to produce a high flow rate with a minimal level of hemolysis and shear. Accordingly, there is an ongoing need to provide alternative blood pumping systems for percutaneous circulatory support device.

### SUMMARY

This disclosure provides design, material, manufacturing method, and use alternatives for medical devices and/or systems, namely percutaneous circulatory support devices that include a diaphragm pump or piston pump. In some examples, a blood pump including multiple, stacked diaphragm pumps may produce a high flow rate with a minimal level of hemolysis, while also being low profile and flexible enough to facilitate percutaneous access and tracking through tortuous anatomy. Percutaneous circulatory support devices and systems including a blood pumping system having multiple, stacked diaphragm pumps are disclosed herein

An example percutaneous circulatory support device system includes a blood pump including a pump housing having a distal end region and a proximal end region, a blood inlet positioned along the distal end region, a blood outlet positioned along the proximal end region and a first diaphragm pump positioned within the pump housing. Further, the first diaphragm pump is configured to draw blood into the pump inlet and pump blood out of the blood outlet.

Alternatively or additionally to any of the embodiments above, wherein the blood inlet is configured to be positioned in the left ventricle.

Alternatively or additionally to any of the embodiments above, wherein the blood outlet is configured to be positioned in the ascending aorta.

Alternatively or additionally to any of the embodiments above, further comprising a second diaphragm pump positioned within the pump housing, and wherein the first diaphragm pump and the second diaphragm pump are aligned along a longitudinal axis of the pump housing.

Alternatively or additionally to any of the embodiments above, further comprising a first solenoid positioned along the distal end region of the pump housing, a second solenoid positioned along the proximal end region of the pump housing and a drive shaft coupled to the first solenoid, the second solenoid, the first diaphragm pump and the second diaphragm pump.

Alternatively or additionally to any of the embodiments above, wherein the first solenoid and the second solenoid are configured to actuate the drive shaft, and wherein actuation of the drive shaft pumps blood through the first diaphragm pump and the second diaphragm pump.

Alternatively or additionally to any of the embodiments above, wherein the first diaphragm pump and the second diaphragm pump are configured to pump blood simultaneously.

Alternatively or additionally to any of the embodiments above, wherein the first diaphragm pump includes a first diaphragm positioned within a first pump chamber of the first diaphragm pump, and a second diaphragm positioned within a second pump chamber of the first diaphragm pump, and wherein the first pump chamber is fluidly sealed from the second pump chamber.

Alternatively or additionally to any of the embodiments above, wherein the first diaphragm pump includes a plurality of inlet valves, and wherein the housing includes a first inlet aperture, and wherein the first inlet aperture is aligned with the plurality of inlet valves.

Alternatively or additionally to any of the embodiments above, wherein the first diaphragm pump includes a plurality of outlet valves, and wherein the housing includes an outlet channel, and wherein the outlet channel is configured to received blood exiting the plurality of outlet valves.

Alternatively or additionally to any of the embodiments above, wherein the outlet channel is in fluid communication with the blood outlet.

Alternatively or additionally to any of the embodiments above, further comprising a blood inlet tube having a proximal end coupled to the distal end region of the pump housing, and wherein the blood inlet is positioned on a distal end region of the blood inlet tube.

Alternatively or additionally to any of the embodiments above, wherein the pump housing is configured to be positioned proximal to the aortic valve, and wherein the blood inlet tube is configured to extend through the aortic valve, and wherein the blood inlet is configured to be positioned in the left ventricle.

Alternatively or additionally to any of the embodiments above, wherein the blood inlet tube includes a first lumen and a second lumen, and wherein the first lumen is configured to transport blood from the left ventricle to the pump housing, and wherein the second lumen is configured to accept a guidewire extending therein.

Another example percutaneous circulatory support device system includes a console including a processor, a cardiac pump including a controller coupled to the console, wherein the cardiac pump includes a pump housing having a distal end region and a proximal end region, a blood inlet positioned along the distal end region, a blood outlet positioned along the proximal end region and a plurality of diaphragm pumps positioned within the pump housing. Further, the plurality of diaphragm pumps are configured to draw blood into the pump inlet and pump blood out of the blood outlet.

Alternatively or additionally to any of the embodiments above, wherein the blood inlet is configured to be positioned in the left ventricle.

Alternatively or additionally to any of the embodiments above, wherein the blood outlet is configured to be positioned in the ascending aorta.

Alternatively or additionally to any of the embodiments above, further comprising a first solenoid positioned along the distal end region of the pump housing, a second solenoid positioned along the proximal end region of the pump housing and a drive shaft coupled to the first solenoid, the second solenoid, and the plurality of diaphragm pumps.

Alternatively or additionally to any of the embodiments above, wherein the plurality of diaphragm pumps are configured to pump blood simultaneously.

An example method for positioning a cardiac pump in a heart includes advancing a cardiac pump adjacent to a left ventricle of the heart. Further, the cardiac pump device includes a blood pump including a pump housing having a distal end region and a proximal end region, a blood inlet positioned along the distal end region, a blood outlet positioned along the proximal end region and a first diaphragm pump positioned within the medial region. The method further includes positioning the blood inlet in the left ventricle and positioning the blood outlet in the ascending aorta of the heart.

Another example percutaneous circulatory support device includes a catheter shaft and a blood pump positioned at a distal end region of the catheter shaft. The blood pump includes a pump housing having a distal end region and a proximal end region, a blood inlet positioned along the distal end region, a blood outlet positioned along the proximal end region and a piston pump positioned within the pump housing. Further, the piston pump includes a piston configured to shift between a first position and a second position and shifting the piston between the first position and the second position draws blood into the pump inlet and pumps blood out of the blood outlet.

Another example percutaneous circulatory support device includes a catheter shaft and a blood pump positioned at a distal end region of the catheter shaft. The blood pump includes a pump housing having a distal end region and a proximal end region, a blood inlet positioned along the distal end region, a blood outlet positioned along the proximal end region and a drive shaft positioned within the pump housing. Further, the drive shaft is configured to axially reciprocate between a first position and a second position to draw blood into the pump inlet and pump blood out of the blood outlet.

Alternatively or additionally to any of the embodiments above, the blood pump includes a piston fixedly attached to the drive shaft, wherein the piston is configured to reciprocate in the pump housing via reciprocation of the drive shaft.

Alternatively or additionally to any of the embodiments above, the blood pump includes a diaphragm attached to the drive shaft, wherein the diaphragm is configured to reciprocate in the pump housing via reciprocation of the drive shaft.

Alternatively or additionally to any of the embodiments above, the blood pump includes a first solenoid coupled to the drive shaft, wherein energizing the first solenoid moves the drive shaft in a first axial direction.

Alternatively or additionally to any of the embodiments above, the blood pump includes a second solenoid coupled to the drive shaft, wherein energizing the second solenoid moves the drive shaft in a second axial direction.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

FIG. 1 depicts a percutaneous circulatory support system, including a circulatory support device and its relative position in a heart of a patient;

FIG. 2 is a schematic block diagram of a console management system;

FIG. 3 depicts a portion of the circulatory support system shown in FIG. 1 positioned in the heart of a patient;

FIG. 4 depicts a portion of a circulatory support system;

FIG. 5 depicts a diaphragm pump;

FIG. 6 depicts a diaphragm pump;

FIG. 7 is a cross-section of a diaphragm pump in a first pumping configuration;

FIG. 8 is a cross-section of a diaphragm pump in a second pumping configuration;

FIG. 9 is a cross-section of a cardiac pump; and

FIG. 10 depicts a portion of a circulatory support system positioned in the heart of a patient.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For this disclosure, the discussion herein is directed toward a percutaneous circulatory support device positioned proximal to an aortic valve and will be so described in the interest of brevity. This, however, is not intended to be limiting as the skilled person will recognize that the following discussion may also apply to other heart valves, with no or minimal changes to the structure and/or scope of the disclosure.

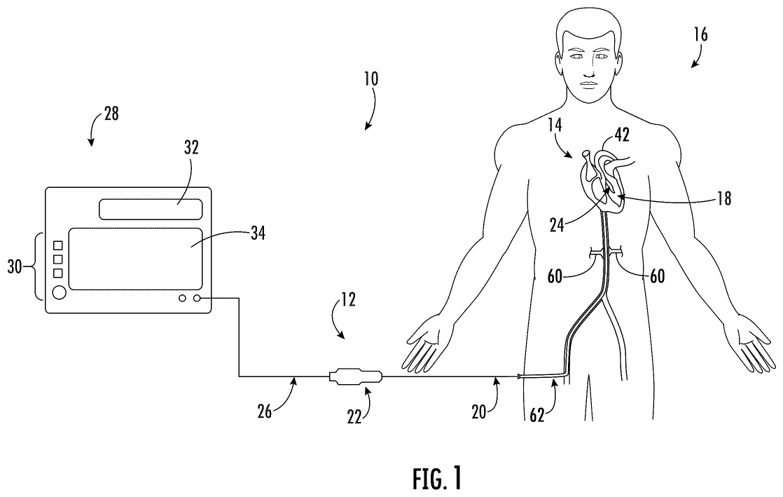

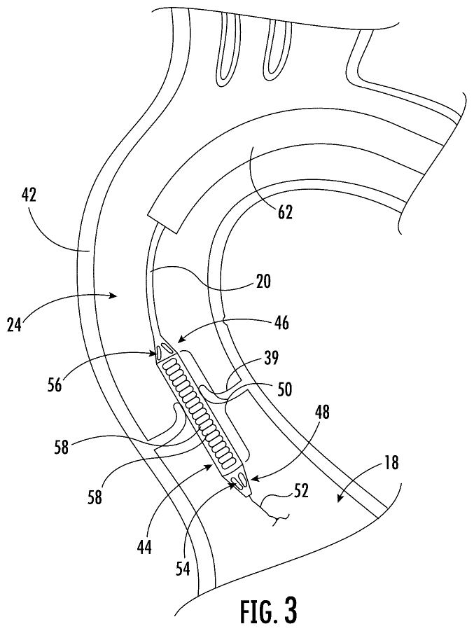

FIG. 1 illustrates an example percutaneous circulatory support system 10 including a circulatory support device 12 positioned in the heart 14 of a patient 16. The circulatory support device 12 may include a flexible elongated catheter shaft 20 having a first end attached to a controller 22 and a second end attached to a blood pump 24. FIG. 1 illustrates the blood pump 24 positioned in the ascending aorta 42 and/or left ventricle 18 of the patient 16. The blood pump 24 may be delivered (e.g., tracked) to the aortic arch and/or ventricle 18 percutaneously over a guidewire 52 (shown in FIG. 3) and/or through a guide sheath 62. For example, the catheter shaft 20 and blood pump 24 may be tracked over a guidewire 52 and/or through the guide sheath 62 through the femoral artery, past the renal arteries 60 and the descending aorta, over the aortic arch, through the ascending aorta 42, past the aortic valve 39 (shown in FIG. 3) and into the left ventricle 18. In some examples, a distal end of the guide sheath 62 may be positioned in the ascending aorta 42 while the guidewire 52 may extend into the left ventricle 18.

Additionally, while FIG. 1 illustrates the blood pump 24 being delivered to the aortic arch and/or ventricle via a femoral approach, other delivery options are contemplated. For example, the blood pump 24 may be delivery via a left or right side trans-axillary approach. One benefit to utilizing a trans-axillary approach is that the patient may remain in an ambulatory condition whereby the patient may be able to walk around and not remain bedridden.

An example of the position of the distal end of the delivery sheath 62 is shown in FIG. 3. In some examples, it can be appreciated that the blood pump 24 may be tracked over a guidewire 52 which extends through a manifold attached to a proximal end of the delivery sheath 62. The blood pump 24 may then be tracked over the guidewire 52 and through the delivery sheath 62 to a position in the patient's heart. However, in other examples, a guidewire may be utilized to position the delivery sheath 62, after which the guidewire may be removed from the patient. In these examples, the blood pump 24 may be tracked through the delivery sheath 62 without a guidewire, to a delivery position in the patient's heart.

FIG. 1 further illustrates that the controller 22 may include a distal end region attached to the catheter shaft 20 and a proximal end region attached to an electrical power cable 26. The electrical power cable 26 may include a distal end region connected to a console 28. It can be appreciated that the controller 22 may include one or more actuators (e.g., buttons, levers, dials, switches, etc.) designed to permit a clinician to control various functions of the blood pump 24. For example, a clinician may be able to control the speed of the motor and/or an impeller located in the blood pump 24 via actuation of one or more actuators located on the controller 22.

Additionally, FIG. 1 illustrates that the console 28 may include one or more control knobs (e.g., buttons, knobs, dials, etc.) 30 and/or one or more displays. For example, FIG. 1 illustrates the console 28 may include a first display 32 and a second display 34. It can be appreciated that the console 28 may include more than two displays. Additionally, while FIG. 1 illustrates the first display 32 and the second display 34 integrated into the console 28, it is contemplated that the circulatory system 10 may be designed such that the first display 32, the second display 34 or both the first display 32 and the second display 34 are separate, distinct components of the circulatory system 10. In other words, the first display 32, the second display 34 or both the first display 32 and the second display 34 may be separate stand-alone displays, apart from the console 28. In some examples, the first display 32 and the second display 34 may receive their respective data from separate sources.

In some examples, the second display 34 may be designed to attach to the console 28 and/or the first display 32. For example, the first display 32 may be integrated into the console 28 while the second display 34 may be configured to attach to portion of the console 28. In yet other examples, both the first display 32 and the second display 34 may be a separate stand-alone display whereby the second display 34 may be configured to attach to the first display 32, or wherein the first display 32 may be configured to attach to the second display 34.

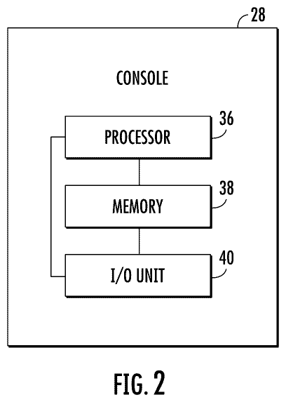

FIG. 2 illustrates that the console 28 may include, among other suitable components, one or more processors 36, memory 38, and an input/output (I/O) unit 40. The processor 36 of the console 28 may include a single processor or more than one processor (e.g., a first processor 36 providing data/instructions to the first display 32 and a second processor 36 providing data instructions to a second display 34) working individually or with one another. The processor 36 may be configured to execute instructions, including instructions that may be loaded into the memory 38 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 36 of the console may be configured to execute program instructions. Program instructions may include, for example, firmware, microcode or application code that is executed by the processor 36, a microprocessor and/or microcontroller. The one or more processors 36 may be configured to each manage different functions. They may also be configured to concurrently perform the same functions (e.g., redundant system). Further yet, they may be configured such that a first processor 36 performs a given function and second processor 36 checks the result of the function of the first processor 36 for correctness (e.g., command-monitor system).

In some examples, the first display 32 may be controlled primarily by the console's firmware control instructions and, therefore, may require relatively little processing power, relatively few instructions and very simple communication between the processor 36 and the display 32, compared to the second display 34 (e.g., a touch screen display 34), which may be controlled primarily by an embedded computer with a flexible and relatively complex communication protocol.

The memory 38 of the console 28 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory 38 may be or may include a non-transitory computer readable medium.

The I/O units 40 of the console 28 may include a single I/O component or more than one I/O component each working individually or with one another. Example I/O units 40 may be any type of communication port configured to communicate with other components of the circulatory system 10. Example types of I/O units 40 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

FIG. 3 illustrates the blood pump 24 of the percutaneous circulatory system 10 extending from the ascending aorta 42 to the left ventricle 18 of a patient 16. The blood pump 24 may include a pump housing 44 having a proximal end region 46, a distal end region 48 and a medial region 50 extending between the proximal end region 46 and the distal end region 48. The proximal end region 46 of the pump housing 44 may be attached to a distal end of the catheter shaft 20. FIG. 3 illustrates that, in some examples, the blood pump 24 may be positioned within the heart 14 such that the housing 44 passes through the aortic valve 39, whereby the distal end region 48 of the pump housing 44 may be positioned within the left ventricle 18. In other instances, the blood pump 24 may be positioned in the left ventricle 18 with a blood outflow tube extending through the aortic valve 39 into the aorta 42. In yet other instances, the blood pump 24 may be positioned in the aorta 42 with a blood inflow tube extending through the aortic valve 39 into the left ventricle 18. As discussed herein, the blood pump 24 may be tracked over a guidewire to its position illustrated in FIG. 3.

FIG. 3 further illustrates that the blood pump 24 may include one or more blood inlets 54 located on a distal end region 48 of the pump housing 44. Additionally, FIG. 3 illustrates that the blood pump 24 may include one or more blood outlets 56 located on a proximal end region 46 of the pump housing 44. In some examples, the blood pump 24 may be positioned within the heart 14 (shown in FIG. 1) such that the one or more blood inlets 54 positioned along the distal end region 48 of the pump housing 44 may be positioned in the left ventricle 18 (or a blood inflow conduit in fluid communication with the one or more blood inlets 54) and the one or more blood outlets 56 located on the proximal end region 46 of the pump housing 44 may be positioned in the ascending aorta 42 (or a blood outflow conduit in fluid communication with the one or more blood outlets 56). Additionally, FIG. 3 illustrates that the medial portion 50 of the blood pump 44 may extend through the aortic valve 39.

Additionally, FIG. 3 illustrates that the blood pump 24 may include a diaphragm pump 58, or a plurality of individual diaphragm pumps 58 positioned within the pump housing 44. In some examples, the plurality of individual diaphragm pumps 58 may be positioned within the medial portion 50 of the pump housing 44. FIG. 3 illustrates that each of the individual diaphragm pumps 58 (the terms “diaphragm pump” or “pump” may be used interchangeably herein) may be “stacked” adjacent to one another (e.g., positioned adjacent to one another) along the longitudinal axis of the pump housing 44.

In some examples, the blood pump 24 may include a single diaphragm pump, 2 or more diaphragm pumps, or about 4 to about 30 diaphragm pumps, or about 10 to about 20 diaphragm pumps, or about 15 to about 35 diaphragm pumps, or about 20 to about 60 diaphragm pumps, or about 30 to about 55 diaphragm pumps, or about 35 to about 55 diaphragm pumps, or about 25 to about 45 diaphragm pumps, or about 25 to about 45 diaphragm pumps, or about 38 to about 42 diaphragm pumps. As will be described in greater detail, each of the diaphragm pumps 58 may be actuated to draw blood (e.g. pump blood) from the left ventricle 18 (via the one or more blood inlets 54 located on a distal end region 48 of the housing 44) into the ascending aorta 42 (via the one or more blood outlets 56 located along the proximal end region 46 of the pump housing 44). It can be appreciated that the blood pump 24 may be configured to include a given number of diaphragm pumps (e.g., 25-55 diaphragm pumps) which permit the blood pump 24 to be positioned in a relatively straight condition (versus a bent or curved configuration) when implanted and operational.

FIG. 3 further illustrates that the blood pump 24 may be tracked to a position in the heart of a patient via a delivery sheath 62. In other words, the blood pump 24 may be tracked through a lumen of the delivery sheath 62 to a position proximal to the aortic valve 39. The distal end of the delivery sheath 62 may be spaced away from pump housing 44 such that the pump housing 44 may be free from the distal end of the delivery sheath 62 when deployed in the ascending aorta 42 and/or the left ventricle 18. FIG. 3 illustrates that, when deployed from the delivery sheath 62, the proximal end region 46 of the pump housing 44 may be spaced away from the distal end region of the delivery sheath 62.

Additionally, it can be appreciated that the pump housing 44 and each of the diaphragm pumps 58 positioned within the pump housing 44 may be formed from a low durometer, flexible material which may be deformed and/or compressed (radially compressed) to a collapsed delivery configuration for tracking within the lumen of the delivery sheath 62. In other words, the diameter of the lumen of the delivery sheath 62 may be smaller than the outer diameter of the pump housing 44 when the pump housing 44 is free from the delivery sheath 62 and expanded to its deployed configuration. Accordingly, the pump housing 44 (including the diaphragm pumps 58 positioned therein) may be compressed to a collapsed delivery configuration within the delivery sheath 62 when being tracked to the heart, whereby the pump housing 44 and diaphragm pumps 58 expand after being released from the delivery sheath 62 to its deployed configuration for pumping blood. It can be appreciated that forming the diaphragm pumps 58 and the pump housing 44 from a low durometer, flexible material may permit the tracking of the blood pump 24 through the anatomy in an efficient, compressed configuration, while also permitting the individual diaphragm pumps 58 to expand to a larger footprint once released from the delivery sheath 62, and thereby pump a larger volume of blood as compared to pumps having a relatively smaller size.

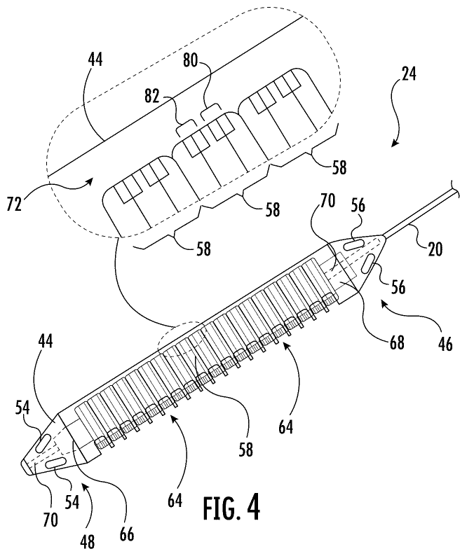

FIG. 4 illustrates a portion of the blood pump 24 described herein including the pump housing 44 and a plurality of individual diaphragm pumps 58 positioned within the medial region 50 (see FIG. 3) of the pump housing 44. FIG. 4 further illustrates the blood inlets 54 (e.g., a plurality of blood inlets 54) located on the distal end region 48 of the pump housing 44 and the one or more blood outlets 56 (e.g., a plurality of blood outlets 56) positioned along the proximal end region 46 of the pump housing 44. FIG. 4 further illustrates that the proximal end region 46 of the pump housing 44 may be coupled to the elongated catheter shaft 20 extending proximally therefrom.

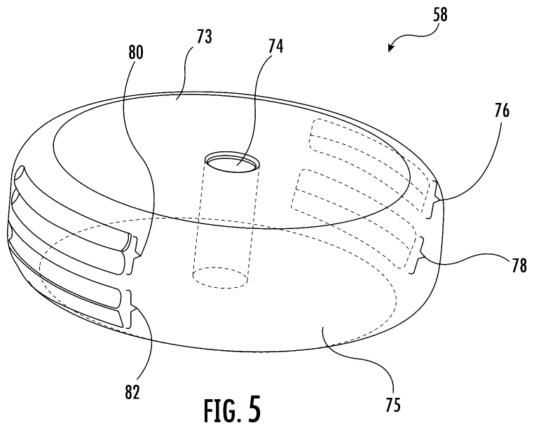

As described herein, FIG. 4 illustrates that a plurality of individual diaphragm pumps 58 may be axially stacked adjacent one another along the longitudinal axis of the pump housing 44. FIG. 5 illustrates that each of the individual diaphragm pumps 58 may include a central aperture 74 extending through the pump 58 from a first surface 73 of the pump 58 to a second surface 75 of the pump 58.

FIG. 4 further illustrates that the blood pump 24 may further include a drive shaft 70 extending from the distal end region 48 of the pump housing 44 to the proximal end region 46 of the pump housing 44. Further, it can be appreciated that the drive shaft 70 may extend through the central aperture 74 of each of the individual diaphragm pumps 58. The drive shaft 70 may be formed from a variety of materials. For example, the drive shaft 70 may be formed from a flexible material that permits the drive shaft 70 to bend, curve, flex, etc., but also permits the drive shaft 70 to have significant axial compression and/or tensile strength. Examples of the materials that may be utilized to form the drive shaft 70 may include polyetheretherketone, polycarbonate, polysulfone, shape memory alloys (e.g., nickel-titanium alloys), etc. A non-limiting list of materials which may be utilized to construct the drive shaft 70 is disclosed herein.

Further, the drive shaft 70 may include a central lumen which may permit the guidewire 52 (shown in FIG. 3) to extend therein. Further yet, in some examples, the central lumen of the drive shaft 70 may be in fluid communication with the lumen of the catheter shaft 20. It can be appreciated that this configuration may permit the guidewire 52 to extend from a position outside the patient, through the lumen of the catheter shaft 20 and through the lumen of the drive shaft 70.

FIG. 4 further illustrates that the blood pump 24 may further include a first solenoid element 66 positioned along the distal end region 48 of the pump housing 44 and/or a second solenoid element 68 positioned along the proximal end region 46 of the pump housing 44. FIG. 4 further illustrates that each of the first solenoid element 66 and the second solenoid element 68 may include a central aperture which permits the drive shaft 70 to extend therethrough. As will be discussed in greater detail herein, each of the first solenoid element 66 and the second solenoid element 68 may be configured to actuate the drive shaft 70 in a reciprocating motion along the longitudinal axis of the pump housing 44 (e.g., in an axially back-and-forth motion). In other examples, the blood pump 24 may include a motor having components such as cams, screws or a rack and pinion system configured to actuate the drive shaft 70 in a reciprocating motion along the longitudinal axis of the pump housing 44 (e.g., in an axially back-and-forth motion).

FIG. 4 further illustrates that the pump housing 44 may further include a plurality of inlet apertures 64 extending from the distal end region 48 of the pump housing 44 to the proximal end region 46 of the pump housing 44, or otherwise arranged along a length of the pump housing 44 between the distal end region 48 of the pump housing 44 and the proximal end region 46 of the pump housing 44. It can be appreciated that each of the individual inlet apertures 64 may align with a first pair of one-way inlet valves 76 (shown in FIG. 5) and a second pair of one-way inlet valves 78 (shown in FIG. 5) of an individual diaphragm pump 58. FIG. 5 illustrates that each individual diaphragm pump 58 may include a first pair of one-way inlet valves 76 and a second pair of one-way inlet valves 78, each of which is configured to permit blood to flow from outside of the diaphragm pump 58 into an inner chamber of the pump.

Further, the detailed view of FIG. 4 and FIG. 5 illustrate that each individual diaphragm pump 58 may include at least one one-way outlet valve, or a plurality of one-way outlet valves, such as a first pair of one-way outlet valves 80 and a second pair of one-way outlet valves 82, each of which may be configured to permit blood to flow from the inner chamber of the diaphragm pump 58 to a location outside the diaphragm pump 58. Additionally, the detailed view of FIG. 4 illustrates that the pump housing 44 may include an outlet channel 72 extending along the medial region 50 (shown in FIG. 3) of the pump housing 44, whereby the outlet channel 72 may be configured to channel blood flowing out of the outlet valve(s), such as first pair of outlet valves 80 and the second pair of outlet valves 82, within the pump housing 44 and out of the blood outlets 56 located along the proximal end region 46 of the pump housing 44. It can be appreciated that the outlet channel 72 may be in fluid communication with the blood outlets 56.

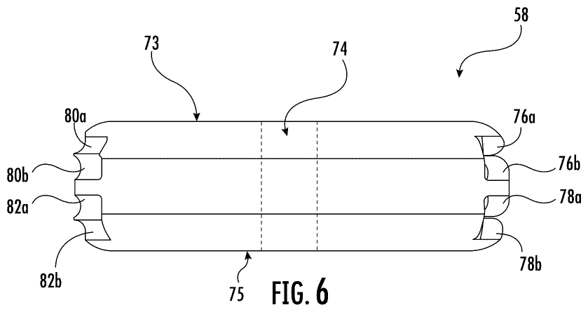

FIG. 6 illustrates that each individual diaphragm pump 58 may include at least one one-way inlet valve, or a plurality of one-way inlet valves, such as a first inlet valve 76a, a second inlet valve 76b, a third inlet valve 78a, and a fourth inlet valve 78b. As illustrated in FIG. 6, each of the first inlet valve 76a, the second inlet valve 76b, the third inlet valve 78a, and the fourth inlet valve 78b may be vertically aligned with one another along a lateral extent of the diaphragm pump 58. Additionally, FIG. 6 illustrates that each individual diaphragm pump 58 may include a first outlet valve 80a, a second inlet valve 80b, a third outlet valve 82a, and a fourth outlet valve 82b. As illustrated in FIG. 6, each of the first outlet valve 80a, the second inlet valve 80b, the third outlet valve 82a, and the fourth outlet valve 82b may be circumferentially aligned with one another along a lateral extent of the diaphragm pump 58, whereby the pump outlet valves 80a, 80b, 82a, 82b are circumferentially positioned approximately 180 degrees opposite to the pump inlet valves 76a, 76b, 78a, 78b. FIG. 6 also illustrates that central aperture of 74 extending through the pump 58 from a first surface 73 of the pump 58 to a second surface 75 of the pump 58.

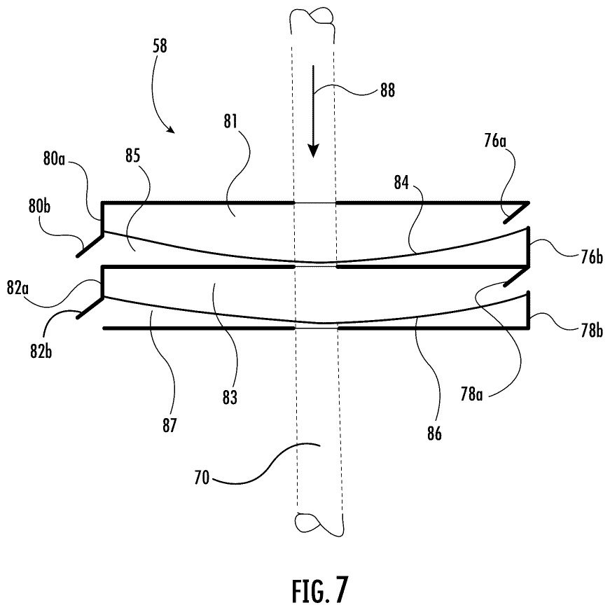

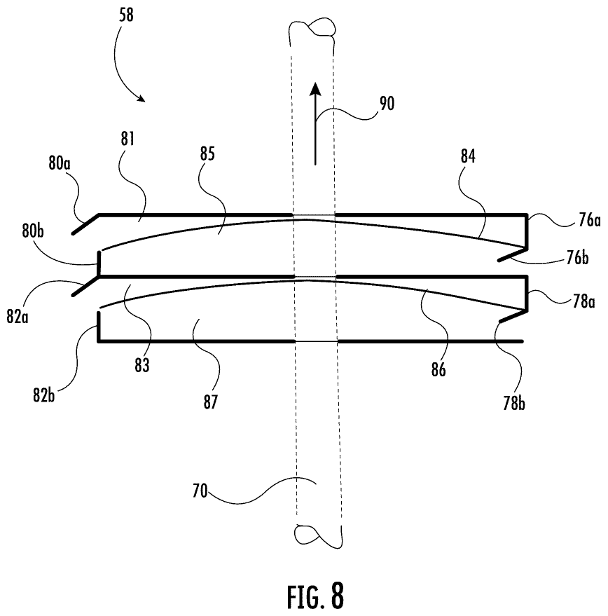

FIGS. 7-8 illustrate the mechanical pumping action of an individual diaphragm pump 58 of the blood pump 24. Each individual diaphragm pump 58 may include one or more movable diaphragms, or a plurality of moveable diaphragms, for pumping blood from a respective pump inlet valve to a respective pump outlet valve. Each individual diaphragm pump 58 may utilize a combination of the reciprocating action of two centralized diaphragms 84, 86 together with one-way pump inlet valves 76a, 76b, 78a, 78b (e.g., flap valves) and the one-way pump outlet valves 80a, 80b, 82a, 82b (e.g., flap valves) on either side of the diaphragms 84, 86 to pump blood from the blood inlets 54, through the pump housing 44 and out of the blood outlets 56.

FIG. 7 illustrates the drive shaft 70 being actuated through the first half of a reciprocating pumping cycle (e.g., a distal stroke portion of a reciprocating pumping cycle). The downward arrow 88 in FIG. 7 illustrates the direction (e.g., distal direction) that the drive shaft 70 is moving during this portion of the reciprocating pumping cycle of the diaphragm pump 58. It is noted that in other instances, the direction of arrow 88 in FIG. 7 may be in the proximal direction depending on the orientation of the diaphragm pump 58. As discussed herein, the drive shaft 70 may be actuated via the action of the first solenoid element 66 (shown in FIG. 4) and the second solenoid element 68 (shown in FIG. 4). In other words, actuation of the first solenoid element 66 and/or the second solenoid element 68 may move the drive shaft 70 in an axially reciprocating manner.

It can be appreciated that the first solenoid element 66 and the second solenoid element 68 may be electromagnets that convert electrical energy into mechanical motion. For example, the first solenoid element 66 and the second solenoid element 68 may be electromagnets that convert electrical energy into mechanical motion of the drive shaft 70 (e.g., axial motion of the drive shaft 70). It can be appreciated that, in some examples, electrical energy may be transferred from a power source located outside the patient (e.g., a power source located in the console 28) to the first solenoid element 66 and/or the second solenoid element 68 via electrical wires extending through the catheter shaft 20.

In some examples, each of the first solenoid element 66 and the second solenoid element 68 (and correspondingly, the drive shaft 70) may be configured to cycle at a given frequency. For example, each of the first solenoid element 66 and the second solenoid element 68 (and correspondingly, the drive shaft 70) may be configured to cycle at about 20 Hz to about 200 Hz, or about 40 Hz to about 160 Hz, or about 60 Hz to about 140 Hz, or about 80 Hz to about 120 Hz, or about 100 Hz. Further, in some examples, the blood pump 24 may be configured to output a volumetric flowrate of 4.0 L/min or more, 5.0 L/min or more, about 4.5 L/min to about 6.5 L/min, or about 5.5 L/min when the first solenoid element 66 and the second solenoid element 68 are cycling between about 80 Hz to about 120 Hz, or about 100 Hz.

Alternatively, in other examples, the blood pump 24 may include a single solenoid element combined with a return spring, whereby the single solenoid element powers the actuation of a drive shaft 70 in one direction when energized, while a return spring powers the actuation of the drive shaft 70 in the opposite direction when the solenoid element is deenergized. For example, a single solenoid element may actuate the drive shaft 70 in an axially distal direction when energized, while a return spring actuates the drive shaft 70 in an axially proximal direction when the solenoid element is deenergized. In another example, a single solenoid element may actuate the drive shaft 70 in an axially proximal direction when energized, while a return spring actuates the drive shaft 70 in an axially distal direction when the solenoid element is deenergized. The actuation of the drive shaft 70 via a solenoid element followed by the energy release of the return spring may power the entire pumping cycle of the diaphragm pump 58.

FIG. 7 further illustrates that the diaphragm pump 58 may include a first diaphragm 84 and/or a second diaphragm 86, each of which is attached to the drive shaft 70. The first diaphragm 84 may divide a first pump chamber 81 from a second pump chamber 85, while the second diaphragm 86 may divide a third pump chamber 83 from a fourth pump chamber 87 all within a single diaphragm pump 58. Additionally, in other examples, the diaphragm pump 58 may include only a single diaphragm which is attached to the drive shaft 70. In yet other examples, the diaphragm pump 58 may include a plurality of diaphragms, each positioned within a respective pump chamber, all within the same diaphragm pump. For example, the diaphragm pump 58 may include 2 or more diaphragms, 3 or more diaphragms, 4 or more diaphragms, etc., whereby all the diaphragms positioned within a given diaphragm pump 58 are coupled to the drive shaft 70 and pump in unison. In other words, it is contemplated that a single diaphragm pump 58 may include a plurality of diaphragms moving in unison with the actuation of the drive shaft 70 in an axially reciprocating manner.

FIG. 7 further illustrates that when the drive shaft 70 is actuated in a first direction (as shown by arrow 88), the first diaphragm 84 and the second diaphragm 86 may be driven in a first direction, thereby decreasing the pressure in the first pump chamber 81 and the third pump chamber 83. Further, the pressure differential caused by the movement of the first diaphragm 84 may cause the inlet valve 76a to open, while the pressure differential caused by the movement of the second diaphragm 86 may cause the inlet valve 78a to open. The opening of the inlet valve 76a and the inlet valve 78a permits blood to flow into the first pump chamber 81 and the third pump chamber 83. It can be appreciated that the pressure differential which opens the inlet valves 76a, 78a may cause the outlet valves 80a, 82a to close.

Further, simultaneous with the opening of the inlet valve 76a and the inlet valve 78a, the movement of the first diaphragm 84 may push blood out of the second pump chamber 85 through the outlet valve 80b, while the movement of the second diaphragm 86 may push blood out of the fourth pump chamber 87 through the outlet valve 82b. Further, the force of the blood being pushed out of the second pump chamber 85 may force the inlet valve 76b to close, while the force of the blood being pushed out of the fourth pump chamber 87 may force the inlet valve 78b to close.

FIG. 8 illustrates the drive shaft 70 being actuated through the second half of a reciprocating pumping cycle (e.g., a proximal stroke portion of a reciprocating pumping cycle). The upward arrow 90 in FIG. 8 illustrates the direction (e.g., proximal direction) that the drive shaft 70 is moving during this portion of the reciprocating pumping cycle of the diaphragm pump 58. It is noted that in other instances, the direction of arrow 90 in FIG. 8 may be in the distal direction depending on the orientation of the diaphragm pump 58. As discussed herein, the drive shaft 70 may be actuated via the action of the first solenoid element 66 (shown in FIG. 4) and the second solenoid element 68 (shown in FIG. 4). In other words, actuation of the first solenoid element 66 and/or the second solenoid element 68 may move the drive shaft 70 in an axially reciprocating manner. For example, energizing the first solenoid element 66 while deenergizing the second solenoid element 68 may axially move the drive shaft 70 in a first direction (e.g., the direction of arrow 88 in FIG. 7), and deenergizing the first solenoid element 66 while energizing the second solenoid element 68 may axially move the drive shaft 70 in a second direction (e.g., the direction of arrow 90 in FIG. 8), opposite the first direction. It is noted that in some instances, the roles of the first and second solenoids 66, 68 may be reversed, such that energizing the first solenoid element 66 while deenergizing the second solenoid element 68 may axially move the drive shaft 70 in the second direction (e.g., the direction of arrow 90 in FIG. 8), and deenergizing the first solenoid element 66 while energizing the second solenoid element 68 may axially move the drive shaft 70 in the first direction (e.g., the direction of arrow 88 in FIG. 7), opposite the second direction.

It can be appreciated that the first solenoid element 66 and the second solenoid element 68 may be electromagnets that convert electrical energy into mechanical motion. For example, the first solenoid element 66 and the second solenoid element 68 may be electromagnets that convert electrical energy into mechanical motion of the drive shaft 70. It can be appreciated that, in some examples, electrical energy may be transferred from a power source located outside the patient (e.g., a power source located in the console 28) to the first solenoid element 66 and/or the second solenoid element 68 via electrical wires extending through the catheter shaft 20.

Similar to that described with respect to FIG. 7, FIG. 8 further illustrates that the first diaphragm 84 may divide a first pump chamber 81 from a second pump chamber 85, while the second diaphragm 86 may divide a third pump chamber 83 from a fourth pump chamber 87 all within a single diaphragm pump 58.

FIG. 8 further illustrates that when the drive shaft 70 is actuated in the second direction (as shown by arrow 90), the first diaphragm 84 and the second diaphragm 86 may be driven in a second direction (opposite the first direction discussed with respect to FIG. 7), thereby decreasing the pressure in the second pump chamber 85 and the fourth pump chamber 87. Further, the pressure differential caused by the movement of the first diaphragm 84 may cause the inlet valve 76b to open, while the pressure differential caused by the movement of the second diaphragm 84 may cause the inlet valve 78b to open. The opening of the inlet valve 76b and the inlet valve 78b permits blood to flow into the second pump chamber 85 and the fourth pump chamber 87. It can be appreciated that the pressure differential which opens the inlet valves 76b, 78b may cause the outlet valves 80b, 82b to close.

Further, simultaneous with the opening of the inlet valve 76b and the inlet valve 78b, the movement of the first diaphragm 84 may push blood out of the first pump chamber 81 through the outlet valve 80a, while the movement of the second diaphragm 86 may push blood out of the third pump chamber 83 through the outlet valve 82a. Further, the force of the blood being pushed out of the first pump chamber 81 may force the inlet valve 76a to close, while the force of the blood being pushed out of the third pump chamber 83 may force the inlet valve 78a to close.

It can be appreciated that the mechanical performance of the dual-diaphragm pump configuration 58 described with respect to FIGS. 7-8 permits blood to be pumped through the pump 58 during both the first stroke (e.g., proximal stroke) and the second stroke (e.g., distal stroke) of the pumping cycle. It can be further appreciated that this pumping action may result in a pumping action that may be optimized to handle shear-sensitive fluids such as blood. Further benefits of incorporating a dual-diaphragm pump configuration into the blood pump 24 is that the pump 58 may be essentially leak free, there are no internal seals or components which require lubrication and/or cooling, and the pump 58 may be self-priming.

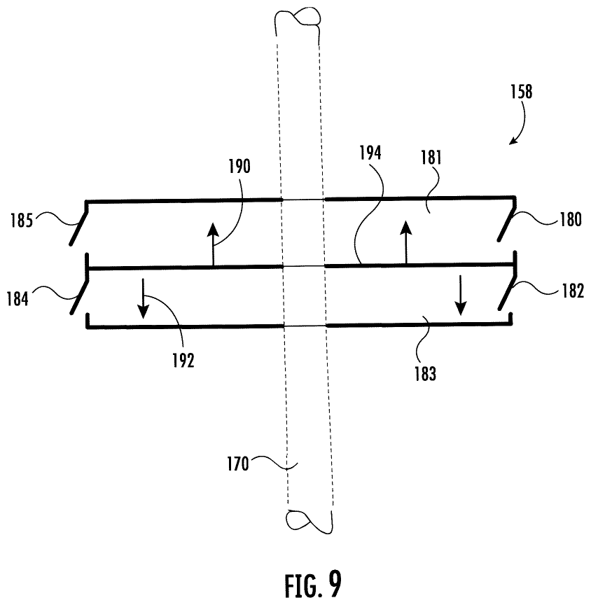

FIG. 9 illustrates another example pump, disclosed as a piston pump 158 which may be utilized to pump blood within the blood pump 24 described herein. In some examples, the piston pump 158 illustrated in FIG. 9 may be substituted for, or alternatively, utilized in combination with the diaphragm pump 58 discussed herein. FIG. 9 illustrates that the piston pump 158 may include a first pump chamber 181 separated from a second pump chamber 183 by a piston 194. Further, FIG. 9 illustrates the piston 194 may be fixedly attached to a drive shaft 170 which extends through a centralized aperture of the piston pump 158. It can be appreciated that the piston 194 may not be fixedly attached to the inner surface of the first pump chamber 181 or the inner surface of the second pump chamber 183, permitting the piston 194 to move up and down (e.g., proximally and distally) within the first pump chamber 181 and the second pump chamber 183.

Further, it can be appreciated that moving the drive shaft 170, and thus the piston 194 attached thereto, in a direction indicated by the arrow 190 (e.g., proximal direction) may open a valve inlet 182 and a valve outlet 185, thereby pulling blood into the second chamber 183 while simultaneously pushing blood out of the first chamber 181. As the piston 194 moves in the first direction indicated by arrow 190, a valve inlet 180 and a valve outlet 184 are forced closed. Additionally, it can be appreciated that moving the drive shaft 170, and thus the piston 194 attached thereto, in a direction indicated by the arrow 192 (e.g., distal direction) may open a valve inlet 180 and a valve outlet 184, thereby pulling blood into the first chamber 181 while simultaneously pushing blood out of the second chamber 183. As the piston 194 moves in the second direction indicated by arrow 192, the valve inlet 182 and the valve outlet 185 are forced closed. It can be appreciated that the mechanical performance of the pump 158 described with respect to FIGS. 9 may permit blood to be pumped through the pump 158 during both the first stroke (e.g., proximal stroke) and the second stroke (e.g., distal stroke) of the pumping cycle.

Similar to that described above, the drive shaft 170 may be actuated via the action of a first solenoid element (such as the first solenoid element shown in FIG. 4) and/or a second solenoid element (such as the second solenoid element 68 shown in FIG. 4). In other words, actuation of a first solenoid element and/or a second solenoid element may move the drive shaft 170 in an axially reciprocating manner. For example, energizing the first solenoid element while deenergizing the second solenoid element may axially move the drive shaft 170 (and thus the piston 194) in a first direction, and deenergizing the first solenoid element while energizing the second solenoid element may axially move the drive shaft 170 (and thus the piston 194) in a second direction, opposite the first direction.

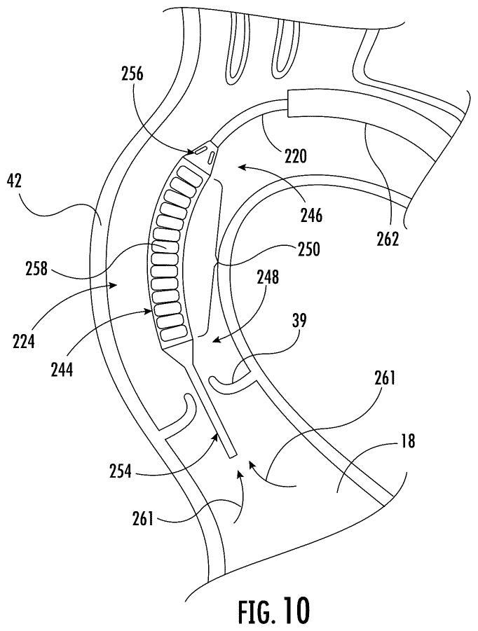

FIG. 10 illustrates another example blood pump 224. The blood pump 224 may be utilized with the percutaneous circulatory system 10 described herein. The blood pump 224 may be similar in form and function to the blood pump 24 described herein. For example, the blood pump 224 may include a pump housing 244 having a proximal end region 246, a distal end region 248 and a medial region 250 extending between the proximal end region 246 and the distal end region 248. The proximal end region 246 of the pump housing 244 may be attached to a distal end of the catheter shaft 220. FIG. 10 illustrates that, in some examples, the blood pump 224 may be positioned within the heart 14 (shown in FIG. 1) such that the housing 244 passes through the aortic valve 39, whereby the distal end region 248 of the pump housing 244 may be positioned within the left ventricle 18 (shown in FIG. 1). As discussed herein, the blood pump 224 may be tracked over a guidewire to its position illustrated in FIG. 10.

Additionally, FIG. 10 illustrates that the blood pump 224 may include a plurality of individual diaphragm pumps 258 positioned within the medial portion 250 of the pump housing 244. Like that described with respect to the blood pump 24, FIG. 10 illustrates that each of the individual diaphragm pumps 258 may be “stacked” adjacent to one another along the longitudinal axis of the pump housing 244. Further, the diaphragm pumps 258 may be powered by a first solenoid (similar to the solenoid 66 shown in FIG. 4) and a second solenoid (similar to the solenoid 68 shown in FIG. 4).

FIG. 10 further illustrates that the blood pump 224 may include an extended blood inlet tube 254 having a proximal end and a distal end. The proximal end of the inlet tube may be coupled to the distal end region 248 of the housing 244. Further, it can be appreciated that the extended inlet tube 254 may be in fluid communication with the housing 244 and/or the individual diaphragm pumps 258. For example, the extended inlet tube 254 may include one or more lumens extending along its length, whereby blood may enter the distal end of the extended inlet tube 254 from the left ventricle, pass through a blood lumen within the extended inlet tube 254 to the diaphragm pumps 258 and get pumped into the ascending aorta 42 through the blood outlets 256. The flow of blood from the left ventricle into the distal end of the extended inlet tube 254 is depicted by the arrows 261 in FIG. 10.

Further, it can be appreciated that the extended inlet tube 254 may permit the pump housing 244 to remain in the ascending aorta 42 (proximal to the aortic valve 39) while the body of the extended inlet tube 254 extends through the aortic valve 39 to position the distal end of the extended inlet tube 254 in the left ventricle 18. This may be beneficial as the overall size of the pump housing 44 (including the individual diaphragm pumps 258) may be increased to improve blood flow rates. Additionally, having only the extended inlet tube 254 extending through the aortic valve 39 may be further beneficial as it minimizes contact of the housing 44 with the aortic valve 39.

As discussed herein, the extended inlet tube 254 may include multiple lumens extending therein. It can be appreciated that, in some examples, the extended inlet tube 254 may include one lumen which is dedicated as a blood inlet to receive blood entering the extended inlet tube 254 from the left ventricle 18, while another lumen of the extended inlet tube 254 may be a dedicated guidewire lumen.

The materials that can be used for the various components of the system 10 may include those commonly associated with medical devices. For simplicity purposes, the following discussion refers to the various components (including the blood pump 24, 224 and components thereof) of the system 10.

The various components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316L V stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the various components of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the various components of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the various components of the system 10 to achieve the same result.

In some embodiments, a degree of Magnetic Resonance Imaging (MRI) compatibility is imparted into the various components of the system 10 For example, the various components of the system 10, or portions thereof, may be made of a material that does not substantially distort the image and create substantial artifacts (e.g., gaps in the image). Certain ferromagnetic materials, for example, may not be suitable because they may create artifacts in an MRI image. The various components of the system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support device, comprising:
a blood pump including a pump housing having a distal end region and a proximal end region;
a blood inlet positioned along the distal end region;
a blood outlet positioned along the proximal end region; and
a first diaphragm pump positioned within the pump housing;
wherein the first diaphragm pump is configured to draw blood into the pump inlet and pump blood out of the blood outlet.

2. The percutaneous circulatory support device of claim 1, wherein the blood inlet is configured to be positioned in the left ventricle.

3. The percutaneous circulatory support device of claim 2, wherein the blood outlet is configured to be positioned in the ascending aorta.

4. The percutaneous circulatory support device of claim 1, further comprising a second diaphragm pump positioned within the pump housing, and wherein the first diaphragm pump and the second diaphragm pump are aligned along a longitudinal axis of the pump housing.

5. The percutaneous circulatory support device of claim 4, further comprising a first solenoid positioned along the distal end region of the pump housing, a second solenoid positioned along the proximal end region of the pump housing and a drive shaft coupled to the first solenoid, the second solenoid, the first diaphragm pump and the second diaphragm pump.

6. The percutaneous circulatory support device of claim 5, wherein the first solenoid and the second solenoid are configured to actuate the drive shaft, and wherein actuation of the drive shaft pumps blood through the first diaphragm pump and the second diaphragm pump.

7. The percutaneous circulatory support device of claim 6, wherein the first diaphragm pump and the second diaphragm pump are configured to pump blood simultaneously.

8. The percutaneous circulatory support device of claim 1, wherein the first diaphragm pump includes a first diaphragm positioned within a first pump chamber of the first diaphragm pump, and a second diaphragm positioned within a second pump chamber of the first diaphragm pump, and wherein the first pump chamber is fluidly sealed from the second pump chamber.

9. The percutaneous circulatory support device of claim 1, wherein the first diaphragm pump includes a plurality of inlet valves, and wherein the housing includes a first inlet aperture, and wherein the first inlet aperture is aligned with the plurality of inlet valves.

10. The percutaneous circulatory support device of claim 1, wherein the first diaphragm pump includes a plurality of outlet valves, and wherein the housing includes an outlet channel, and wherein the outlet channel is configured to received blood exiting the plurality of outlet valves.

11. The percutaneous circulatory support device of claim 10, wherein the outlet channel is in fluid communication with the blood outlet.

12. The percutaneous circulatory support device of claim 1, further comprising a blood inlet tube having a proximal end coupled to the distal end region of the pump housing, and wherein the blood inlet is positioned on a distal end region of the blood inlet tube.

13. The percutaneous circulatory support device of claim 12, wherein the pump housing is configured to be positioned proximal to the aortic valve, and wherein the blood inlet tube is configured to extend through the aortic valve, and wherein the blood inlet is configured to be positioned in the left ventricle.

14. The percutaneous circulatory support device of claim 13, wherein the blood inlet tube includes a first lumen and a second lumen, and wherein the first lumen is configured to transport blood from the left ventricle to the pump housing, and wherein the second lumen is configured to accept a guidewire extending therein.

15. A percutaneous circulatory support device system, comprising:
a console including a processor;
a cardiac pump including a controller coupled to the console, wherein the cardiac pump includes a pump housing having a distal end region and a proximal end region;
a blood inlet positioned along the distal end region;
a blood outlet positioned along the proximal end region; and
a plurality of diaphragm pumps positioned within the pump housing;
wherein the plurality of diaphragm pumps are configured to draw blood into the pump inlet and pump blood out of the blood outlet.

16. The percutaneous circulatory support device system of claim 15, wherein the blood inlet is configured to be positioned in the left ventricle.

17. The percutaneous circulatory support device system of claim 16, wherein the blood outlet is configured to be positioned in the ascending aorta.

18. The percutaneous circulatory support device system of claim 15, further comprising a first solenoid positioned along the distal end region of the pump housing, a second solenoid positioned along the proximal end region of the pump housing and a drive shaft coupled to the first solenoid, the second solenoid, and the plurality of diaphragm pumps.

19. The percutaneous circulatory support device system of claim 18, wherein the plurality of diaphragm pumps are configured to pump blood simultaneously.

20. A percutaneous circulatory support device, comprising:
a catheter shaft;
a blood pump positioned at a distal end region of the catheter shaft, the blood pump including a pump housing having a distal end region and a proximal end region;
a blood inlet positioned along the distal end region;
a blood outlet positioned along the proximal end region; and
a piston pump positioned within the pump housing;
wherein the piston pump includes a piston configured to shift between a first position and a second position;
wherein shifting the piston between the first position and the second position draws blood into the pump inlet and pumps blood out of the blood outlet.
